(19) United States
(12) Patent Application Publication
(10) Pub. No.: US 2022/0323023 A1
(43) Pub. Date: Oct. 13, 2022

(54) METHOD FOR DETERMINING RESPIRATORY RATE

(71) Applicant: SENSORIA ANALYTICS, Vence (FR)

(72) Inventor: Slaheddine ARIDHI, Vence (FR)

(21) Appl. No.: 17/642,671

(22) PCT Filed: Sep. 11, 2020
(86) PCT No.: PCT/EP2020/075569

§ 371 Date: Mar. 11, 2022

(30) Foreign Application Priority Data
Sep. 13, 2019 (FR) FR1910162

(51) Int. Cl. A61B 5/00; A61B 5/0205

(52) U.S. Cl. CPC A61B 5/7278; A61B 5/0205; A61B 5/725; A61B 5/7239; A61B 5/7246; A61B 5/7257; A61B 5/6826; A61B 2562/0204; A61B 2562/0247; A61B 5/02416

(57) ABSTRACT

A method for determining the respiratory rate R of at least one user includes at least the following steps including acquisition of at least one photoplethysmogram S(t), estimation of the heart rate Fc from at least one photoplethysmogram S(t) acquired from the at least one user, obtainment (130) of at least three signals, generation of a signal S8 from the at least three obtained signals, algorithmic processing (140) of each obtained signal and of S8 by at least two algorithms Ai and Aj so as to obtain at least two estimated respiratory rates for each processed signal, computation (150) of at least two intermediate respiratory rates rm and rn from the estimated respiratory rates (Rx.Ai, Rx.Aj), determination (160) of the respiratory rate R by computing the median of the at least two intermediate respiratory rates.

### TECHNICAL FIELD OF THE INVENTION

The present invention relates to the field of devices for collecting medical data, in particular methods for measuring a respiratory rate in a user.

### PRIOR ART

There are many solutions in the prior art aiming to determine the respiratory rate of a user. For example, some solutions use the measurement of the displacement of the rib cage, others use the air pressure at the outlet of the respiratory tract.

One of the main problems of the solutions of the prior art lies in the bulk caused by this type of solutions. Quite often, the user has to carry with him portable equipment that is not easy to adjust or non-portable and bulky equipment.

New solutions have been imagined, yet without success, based on the heart rate to deduce the respiratory rate. These solutions have generally led to a lack of reproducibility, to a very low reliability or still to considerable errors in the respiratory rate thus determined.

The present invention is intended to solve these various problems.

### SUMMARY OF THE INVENTION

The present invention relates to a method for determining the respiratory rate R of at least one user comprising at least the following steps implemented by at least one data processing unit comprising for example at least one amongst a processor and a computer:
a. Preferably, acquisition of at least one photoplethysmogram S(t) of said user, the step of acquiring said photoplethysmogram S(t) comprising at least one amongst the following steps:
   i. Measurement, preferably at a frequency higher than or equal to 50 Hz, of said at least one photoplethysmogram S(t) by a measuring device, preferably worn or intended to be worn by said at least one user;
ii. Reception from at least one database of said at least one photoplethysmogram S(t);
b. Estimation of the heart rate Fc from said at least one photoplethysmogram S(t) acquired from said at least one user;
c. Obtainment of at least three signals (S1, . . . , S7) distinct from each other, each obtained signal (S1, . . . , S7) being a function of said at least one acquired photoplethysmogram S(t) Each of these signals could be referred to as primary signal.
d. Generation of a signal S8 from said at least three obtained signals (S1, . . . , S7). This signal could be referred to as complex signal.
e. Algorithmic processing of each of the at least three obtained signals (S1, . . . , S7) and of the signal S8 by at least two algorithms Ai and Aj, so as to obtain at least two estimated respiratory rates, respectively Rx.Ai and Rx.Aj, for each signal Sx processed respectively by the algorithm Ai and by the algorithm Aj, each signal Sx being taken amongst said at least three obtained signals (S1, . . . , S7) and the signal S8; the at least two algorithms Ai and Aj being distinct from each other;
f. Computation of at least two intermediate respiratory rates rm and rn from the estimated respiratory rates (Rx.Ai, Rx.Aj);
g. Determination of the respiratory rate R by computing the median of said at least two intermediate respiratory rates rm and rn.

The present invention allows determining the respiratory rate of a user from the measurement of a photoplethysmogram in a simple, rapid and reliable manner.

The present invention allows using a simple measuring device and does not require high accuracy in the measurement of the photoplethysmogram.

Thus, the present invention allows saving hardware and computing resources for the determination of a physiological parameter from a simple measurement of a photoplethysmogram over a very short period.

Preferably, the photoplethysmogram acquisition time is longer than 30 seconds, preferably 1 minute and advantageously 2 minutes.

The exploitation of the diversity of respiratory signals that could be extracted from a plethysmography signal and the use of several algorithmic approaches allows generating much information, thus allowing estimating the respiratory rate reliably while minimising the mean absolute error called MAE.

Advantageously, the present invention allows obtaining a value of a respiratory rate of a user from a measurement that could be noisy.

Advantageously, the present invention allows obtaining a value of a respiratory rate of a user from a measuring device that could be inexpensive.

Advantageously, the present invention allows obtaining a reliable value of a respiratory rate of a user.

The present invention also relates to a method for determining the respiratory rate R of at least one user comprising at least the following steps:
a. Acquisition of at least one photoplethysmogram S(t) of said user, preferably at a frequency higher than or equal to 50 Hz;
b. Filtering of said photoplethysmogram, preferably by a band-pass filter, preferably comprised between 0.5 Hz and 4 Hz, so as to generate a first filtered photoplethysmogram;
c. Estimation of the heart rate Fc from the filtered photoplethysmogram, preferably by identifying the maximum value of the power spectral density of the filtered photoplethysmogram;
d. Filtering of the first filtered photoplethysmogram between Fc/20 and 4 Hz so as to generate a second filtered photoplethysmogram;
e. Obtainment of at least three signals distinct from each other taken amongst the following signals S1, S2, S3, S4, S5, S6 and S7:
   i. a signal S1 obtained by Application of a band-pass filter between Fc/20 and Fc/2 to the second filtered photoplethysmogram.
   ii. a signal S2 obtained by:
      determination of the peaks of the beats of the upper envelope of the second filtered photoplethysmogram,
      then by filtering of the signal of the peaks of the beats of the upper envelope by a band-pass filter between Fc/20 and Fc/2.
   iii. a signal S3 obtained by:
      determination of the valleys of the beats of the lower envelope of the second filtered photoplethysmogram;
      then by filtering of the signal of the valleys of the beats of the lower envelope by a band-pass filter between Fc/20 and Fc/2;
   iv. a signal S4 obtained by:
      Generation of a pulse velocity wave VPG by performing a first-order derivative of the photoplethysmogram S(t) relative to the time coordinate of said photoplethysmogram S(t);
      Determination of the positions Pi of the peaks of the pulse velocity wave VPG;
      Obtainment of a signal of variation of the inflection points by reporting values of said photoplethysmogram S(t) at the positions Pi;
      Oversampling of the inflection point variation signal up to a predetermined frequency Fs, preferably generally equivalent to that of the signal S(t);
      Band-pass filtering between the frequencies Fc/20 and Fc/2 of the oversampled signal;
   v. a signal S5 obtained by:
      Location of the inflection points Pi in a systolic rise of a heartbeat of the user by identifying the position of the peaks of the first-order derivative of the photoplethysmogram S(t) relative to the time coordinate of said photoplethysmogram S(t)
      Obtainment of a signal of variation of the inflection points Pi by reporting values of time differences between Pi and Pi−1;
      Oversampling of this signal of intervals of the inflection points Pi up to a predetermined frequency Fs, preferably generally in the same range as S(t);
      Band-pass filtering between the frequencies Fc/20 and Fc/2 of the oversampled signal;
   vi. a signal S6 obtained by:
      determination of the amplitude variations of the second filtered photoplethysmogram,
      then by filtering by a band-pass filter between Fc/20 and Fc/2 of the amplitude variation signal;
   vii. a signal S7 obtained by:
      determination of the valleys of the beats of the lower envelope of the second filtered photoplethysmogram,
      oversampling of the signal of the valleys of the beats of the lower envelope up to a predetermined frequency Fs, preferably generally in the same range as the frequency of the signal S(t);
      First-order differentiation according to the time coordinate of the oversampled signal;
      then by filtering of the derived signal by a band-pass filter between Fc/20 and Fc/2.
f. Generation of a signal S8 from said at least three obtained signals, the step of generating the signal S8 comprising the following steps:
   i. Computation, for each signal of said at least three obtained signals, of a normalised autocorrelation signal;
   ii. Computation of the arithmetic average of the normalised autocorrelation signals.
g. Algorithmic processing of each of the at least three obtained signals and of the signal S8 by at least two algorithms Ai and Aj so as to obtain at least two estimated respiratory rates, respectively Rx.Ai and Rx.Aj, for each signal Sx processed respectively by the algorithm Ai and by the algorithm Aj, each signal Sx being taken amongst said at least three obtained signals and the signal S8; the at least two algorithms Ai and Aj being distinct from each other and being taken amongst at least the following algorithms A1, A2, A3 and A4:
   i. an algorithm A1 comprising at least the following steps applied to the signal Sx:
      Collection of the measurement points corresponding to a crossing of the abscissas by the considered signal, i.e. Sx(t)=0;
      Computation of the average interval Tm between each of the abscissa crossing points
      Computation of Rx.A1 with Rx.A1=60/(2*Tm);
   ii. an algorithm A2 comprising at least the following steps applied to the signal Sx:
      Collection of the measurement points corresponding to the peak of the undulations of the considered signal;
      Computation of the average interval Ts between each of the peak points;
      Computation of Rx.A2 with Rx.A2=60/(Ts);
   iii. an algorithm A3 comprising at least the following steps applied to the signal Sx:
      Determination of the correntropy spectral density by computing the Fourier transform of the centred autocorrentropy of the considered signal;
      Extraction of Rx.A3 by determining the peak of the correntropy spectral density over a predetermined frequency range, preferably between max(Fc/20, 0.05) and min(Fc/2.1) where 0.05 Hz and 1 Hz respectively correspond to respiratory rates of 3 and 60 breaths per minute;
   iv. an algorithm A4 comprising at least the following steps applied to the signal Sx:
      Computation of the autocorrelation signal of the considered signal;
      Collection of the measurement points corresponding to the peak of the undulations of the autocorrelation signal of the considered signal;
      Computation of the average interval Tas between each of the peak points;
      Computation of Rx.A4 with Rx.A4=60/(Tas);
h. Computation of at least two intermediate respiratory rates rm and rn taken amongst at least the following intermediate respiratory rates r1, r2, r3 and r4:
   i. r1 is equal to the median value of the estimated respiratory rates Rx.Ay of a signal Sx, Sx being taken amongst at least the obtained signals;
   ii. r2 corresponds to the top of the histogram formed by the estimated respiratory rates Rx.Ay of each considered signal Sx taken amongst the obtained signals processed throughout each considered algorithm Ay taken amongst A1 to A4;
   iii. r3 is equal to the median value of the estimated respiratory rates Rx.A3 for each considered signal Sx taken amongst the obtained signals processed by the algorithm A3;
   iv. r4 is equal to the median value of the estimated respiratory rates R8.Ay of the signal S8 processed throughout each considered algorithm Ay taken amongst A1 to A4.
i. Determination of the respiratory rate R by computing the median of said at least two intermediate respiratory rates.

The present invention also relates to a computer program product, preferably recorded on a non-transitory medium, comprising instructions, which when performed by at least one amongst a processor and a computer, result in that the at least one amongst the processor and the computer, executes the method according to any one of the preceding claims.

The present invention finally relates to a device for measuring a photoplethysmogram S(t) of a user connected to at least one data processing unit comprising at least one non-transitory memory comprising a computer program product according to the preceding claim, preferably, the measuring device comprises at least one sensor adapted to measure a photoplethysmogram.

The present invention also relates to a method for determining the respiratory rate R of at least one user comprising at least the following steps implemented by at least one data processing unit comprising for example at least one amongst a processor and a computer:
a. Preferably, acquisition of at least one signal depending on the respiratory rate S(t) of said user, the step of acquiring said signal depending on the respiratory rate S(t) comprising at least one amongst the following steps:
   i. Measurement, preferably at a frequency higher than or equal to 50 Hz, of said at least one signal depending on the respiratory rate S(t) by a measuring device, preferably worn or intended to be worn by said at least one user;
   ii. Reception from at least one database of said at least one signal depending on the respiratory rate S(t);
b. Estimation of the heart rate Fc from said at least one signal depending on the respiratory rate S(t) acquired from said at least one user;
c. Obtainment of at least three signals (S1, . . . , S7) distinct from each other, each obtained signal (S1, . . . , S7) being a function of said at least one acquired signal depending on the respiratory rate S(t); Each of these signals could be referred to as primary signal.
d. Generation of a signal S8 from said at least three obtained signals (S1, . . . , S7). This signal could be referred to as complex signal.
e. Algorithmic processing of each of the at least three obtained signals (S1, . . . , S7) and of the signal S8 by at least two algorithms Ai and Aj, so as to obtain at least two estimated respiratory rates, respectively Rx.Ai and Rx.Aj, for each signal Sx processed respectively by the algorithm Ai and by the algorithm Aj, each signal Sx being taken amongst said at least three obtained signals (S1, . . . , S7) and the signal S8; the at least two algorithms Ai and Aj being distinct from each other;
f. Computation of at least two intermediate respiratory rates rm and rn from the estimated respiratory rates (Rx.Ai, Rx.Aj);
g. Determination of the respiratory rate R by computing the median of said at least two intermediate respiratory rates rm and rn.

Preferably, the signal depending on the respiratory rate comprises or consists of a photoplethysmogram.

### BRIEF DESCRIPTION OF THE FIGURES

The aims, objects, as well as the features and advantages of the invention will appear better from the detailed description of embodiments of the latter which is illustrated by the following appended drawings wherein:

FIG. 1 represents a measuring device according to an embodiment of the present invention.

FIG. 2 represents a measuring device according to another embodiment of the present invention.

FIG. 3 represents a diagram schematising an embodiment of the method according to the present invention.

FIG. 4 represents an example of a photoplethysmogram S(t) of a user.

FIG. 5 represents the signal S1 according to an embodiment of the present invention.

FIG. 6 represents the signal S2 according to an embodiment of the present invention.

FIG. 7 represents the signal S3 according to an embodiment of the present invention.

FIG. 8 represents the signal S4 according to an embodiment of the present invention.

FIG. 9 represents the signal S5 according to an embodiment of the present invention.

FIG. 10 represents the signal S6 according to an embodiment of the present invention.

FIG. 11 represents the signal S7 according to an embodiment of the present invention.

FIG. 12 represents the signal S8 according to an embodiment of the present invention.

FIG. 13 represents the computation of an intermediate respiratory rate r2 according to an embodiment of the present invention.

The drawings are provided as examples and do not limit the invention. They consist of schematic principle representations intended to facilitate the understanding of the invention and are not necessarily to the scale of practical applications. In particular, the dimensions do not represent the reality.

### DETAILED DESCRIPTION

Before starting with a detailed review of embodiments of the invention, optional features that could possibly be used in combination or alternatively are set out hereinafter.

According to one embodiment, the method comprises, before the step of estimating the heart rate Fc, a step of filtering said photoplethysmogram, preferably by a band-pass filter, preferably comprised between 0.5 Hz and 4 Hz, so as to generate a first filtered photoplethysmogram.

According to one embodiment, the method comprises, after the step of estimating the heart rate Fc, an additional step of filtering said first photoplethysmogram filtered between Fc/20 and 4 Hz so as to generate a second filtered photoplethysmogram.

According to one embodiment, said at least three obtained distinct signals (S1, . . . , S7) are taken amongst at least the following signals S1, S2, S3, S4, S5, S6 and S7:
a. a signal S1 obtained by:
   i. Application of a band-pass filter between Fc/20 and Fc/2 to the second filtered photoplethysmogram.
b. a signal S2 obtained by:
   i. determination of the peaks of the beats of the upper envelope of the second filtered photoplethysmogram,
   ii. then by filtering of the signal of the peaks of the beats of the upper envelope by a band-pass filter between Fc/20 and Fc/2.
c. a signal S3 obtained by:
   i. determination of the valleys of the beats of the lower envelope of the second filtered photoplethysmogram;
   ii. then by filtering of the signal of the valleys of the beats of the lower envelope by a band-pass filter between Fc/20 and Fc/2;
d. a signal S4 obtained by:
   i. Generation of a pulse velocity wave VPG by performing a first-order derivative of the photoplethysmogram S(t) relative to the time coordinate of said photoplethysmogram S(t);
   ii. Determination of the positions Pi of the peaks of the pulse velocity wave VPG;
   iii. Obtainment of a signal of variation of the inflection points by reporting values of said photoplethysmogram S(t) at the positions Pi;
   iv. Oversampling of the inflection point variation signal up to a predetermined frequency Fs, preferably generally equivalent to that of the signal S(t);
   v. Band-pass filtering between the frequencies Fc/20 and Fc/2 of the oversampled signal;
e. a signal S5 obtained by:
   i. Location of the inflection points Pi in a systolic rise of a heartbeat of the user by identifying the position of the peaks of the first-order derivative of the photoplethysmogram S(t) relative to the time coordinate of said photoplethysmogram S(t)
   ii. Obtainment of a signal of variation of the inflection points Pi by reporting values of time differences between Pi and Pi−1;
   iii. Oversampling of this signal of intervals of the inflection points Pi up to a predetermined frequency Fs, preferably generally in the same range as S(t);
   iv. Band-pass filtering between the frequencies Fc/20 and Fc/2 of the oversampled signal;
f. a signal S6 obtained by:
   i. determination of the amplitude variations of the second filtered photoplethysmogram,
   ii. then by filtering by a band-pass filter between Fc/20 and Fc/2 of the amplitude variation signal;
g. a signal S7 obtained by:
   i. determination of the valleys of the beats of the lower envelope of the second filtered photoplethysmogram,
   ii. oversampling of the signal of the valleys of the beats of the lower envelope up to a predetermined frequency Fs, preferably generally in the same range as the frequency of the signal S(t);
   iii. First-order differentiation according to the time coordinate of the oversampled signal;
   iv. then by filtering of the derived signal by a band-pass filter between Fc/20 and Fc/2.

According to one embodiment, the step of generating a signal S8 from said at least three obtained signals (S1, . . . , S7) comprises at least the following steps:
a. Computation, for each signal amongst said at least three obtained signals (S1, . . . , S7), of a normalised autocorrelation signal;
b. Computation of the arithmetic average of the normalised autocorrelation signals.

According to one embodiment, the at least two algorithms Ai and Aj distinct from each other are taken amongst at least the following algorithms A1, A2, A3 and A4:
a. an algorithm A1 comprising at least the following steps applied to the signal Sx:
   i. Collection of the measurement points corresponding to a crossing of the abscissas by the considered signal, i.e. Sx(t)=0;
   ii. Computation of the average interval Tm between each of the abscissa crossing points
   iii. Computation of Rx.A1 with Rx.A1=60/(2*Tm);
b. an algorithm A2 comprising at least the following steps applied to the signal Sx:
   i. Collection of the measurement points corresponding to the peak of the undulations of the considered signal;
   ii. Computation of the average interval Ts between each of the peak points;
   iii. Computation of Rx.A2 with Rx.A2=60/(Ts);
c. an algorithm A3 comprising at least the following steps applied to the signal Sx:
   i. Determination of the correntropy spectral density by computing the Fourier transform of the centred autocorrentropy of the considered signal;
   ii. Extraction of Rx.A3 by determining the peak of the correntropy spectral density over a predetermined frequency range, preferably between max(Fc/20, 0.05) and min(Fc/2.1) where 0.05 Hz and 1 Hz respectively correspond to respiratory rates of 3 and 60 breaths per minute;
d. an algorithm A4 comprising at least the following steps applied to the signal Sx:
   i. Computation of the autocorrelation signal of the considered signal;
   ii. Collection of the measurement points corresponding to the peak of the undulations of the autocorrelation signal of the considered signal;
   iii. Computation of the average interval Tas between each of the peak points;
   iv. Computation of Rx.A4 with Rx.A4=60/(Tas);

According to one embodiment, the at least two intermediate respiratory rates rm and rn are taken amongst at least the following intermediate respiratory rates r1, r2, r3 and r4:
a. r1 is equal to the median value of the estimated respiratory rates Rx.Ay of a signal Sx, Sx being taken amongst at least the obtained signals;
b. r2 corresponds to the top of the histogram formed by the estimated respiratory rates Rx.Ay of each considered signal Sx taken amongst the obtained signals processed throughout each considered algorithm Ay taken amongst A1 to A4;
c. r3 is equal to the median value of the estimated respiratory rates Rx.A3 for each considered signal Sx taken amongst the obtained signals processed by the algorithm A3;
d. r4 is equal to the median value of the estimated respiratory rates R8.Ay of the signal S8 processed throughout each considered algorithm Ay taken amongst A1 to A4.

According to one embodiment, the method comprises a step of acquiring said at least one photoplethysmogram S(t) of said user, preferably at a frequency higher than or equal to 50 Hz.

According to one embodiment, the step of acquiring said photoplethysmogram S(t) comprises the measurement of said at least one photoplethysmogram S(t) by a device, preferably worn or intended to be worn by said at least one user.

According to one embodiment, the step of obtaining at least three signals (S1, . . . , S7) distinct from each other comprises the obtainment of at least 4, preferably at least 5, advantageously at least 6 and advantageously 7 distinct signals S1, S2, S3, S4, S5, S6 and S7.

Advantageously, the more estimated respiratory signals are considered, the better will be the probability of estimating the respiratory rate with a minimum of error.

According to one embodiment, the algorithmic processing step comprises the use of at least 3 and preferably 4 distinct algorithms A1, A2, A3 and A4 applied to each of the obtained signals (S1, . . . , S7) and the signal S8.

Advantageously, the diversity of the respiratory rate estimation algorithms allows processing all forms of respiratory signals (with or without artifacts) while offering rapid convergence towards the desired result.

According to one embodiment, r1=median (median ((R1.A1, R1.A2, R1.A3, R1.A4)), median ((R2.A1, R2.A2, R2.A3, R2.A4)), median ((R3.A1, R3.A2, R3.A3, A3.A4)), median ((R4.A1, R4.A2, R4.A3, R4.A4)), median ((R5.A1, R5.A2, R5.A3, R5.A4)), median ((R6.A1, R6.A2, R6.A3, R6.A4)), median ((R7.A1, R7.A2, R7.A3, R7.A4))).

Advantageously, this technique favors the estimates that occur around 50% of the time, hence the use of the median.

According to one embodiment, r2 corresponds to the top of the histogram formed by the following values: R1.A1, R1.A2, R1.A3, R1.A4, R2.A1, R2.A2, R2.A3, R2.A4, R3.A1, R3.A2, R3.A3, R3.A4, R4.A1, R4.A2, R4.A3, R4.A4, R5.A1, R5.A2, R5.A3, R5.A4, R6.A1, R6.A2, R6.A3, R6.A4, R7.A1, R7.A2, R7.A3, R7.A4.

Advantageously, this technique favors the most frequent and close estimates.

According to one embodiment, r3=median (R1.A3, R2.A3, R3.A3, R4.A3, R5.A3, R6.A3, R7.A3).

Advantageously, this technique favors estimates based on the frequency analysis of the signals.

According to one embodiment, r4=median (R8.A1, R8.A2, R8.A3, R8.A4).

According to one embodiment, R=median (r1, r2, r3, r4).

According to one embodiment, S8=(S1c+S2c+S3c+S4c+S5c+S6c+S7c)/7, with the signals Sxc corresponding to the normalised autocorrelation signal of the signal Sx taken amongst S1, S2, S3, S4, S5, S6 and S7.

Advantageously, the use of the autocorrelation signals has several advantages: the undulations and the periodicity intrinsic to the signals are highlighted. In addition, the signals are naturally aligned to obtain an effective combination. The normalisation ensures that all of the signals in the combination have the same weight.

According to one embodiment, the device according to the present invention comprises a module for wireless communication with said data processing unit.

According to one embodiment, the device according to the present invention comprises a module for wired communication with said data processing unit.

According to one embodiment, the sensor adapted to measure a photoplethysmogram is a sensor taken amongst at least: an optical sensor, an electrical sensor, a radar sensor, a force sensor, a pressure sensor, a vibration sensor, an audio sensor, a seismic sensor.

According to one embodiment, the device according to the present invention comprises at least one sensor of the respiratory rate of said user taken amongst at least: an optical sensor, an electrical sensor, a radar sensor, a force sensor, a pressure sensor, a vibration sensor, an audio sensor, a seismic sensor.

The present invention relates to a method for determining the respiratory rate R of at least one user. In a particularly tricky manner, this method uses a photoplethysmogram S(t) of said user.

This photoplethysmogram S(t) could be obtained in various manners. In particular, and according to an embodiment of the present invention, it could be obtained by a measuring device comprising at least one sensor selected amongst at least one optical, electrical, radar, pressure force, vibration, audio or still seismic sensor for example.

According to one embodiment, such a measuring device comprises at least one sensor adapted to measure at least one physiological parameter of the user and preferably a parameter related indirectly or not to the breathing of the user.

For example, and preferably, the measuring device may comprise at least one optical sensor and at least one light source. The optical sensor could operate in two manners, either by transparency, i.e. it receives the light rays emitted by the light source and having crossed a portion of the body of the user, a finger for example, or by reflection, i.e. it receives the light rays emitted by the light source and having been reflected by a portion of the body of the user, by his finger for example. Such devices will be described later on.

Preferably, a measuring device according to the present invention is connected to at least one data processing unit at least one non-transitory memory comprising a computer program product. Preferably, this computer program product comprises instructions, which when performed by at least one amongst a processor and a computer, result in that the at least one amongst the processor and the computer, executes the method according to the present invention.

According to one embodiment, the measuring device is in wired and/or wireless communication with said data processing unit.

As will be described later on, and according to a preferred embodiment, the method for determining the respiratory rate R of a user comprises at least the following steps implemented by at least one data processing unit:
a. Acquisition of at least one photoplethysmogram S(t) of said user preferably via a measuring device and/or via a database;
b. A first filtering of the photoplethysmogram S(t) could then be performed;
c. Then, the filtered photoplethysmogram Sf(t) will then be processed so as to obtain a plurality of signals, preferably at least three distinct signals, advantageously at least 4, preferably at least 5, advantageously at least 6 and preferably at least 7, taken amongst signals S1, S2, S3, S4, S5, S6 and S7 described later on;
d. These distinct signals will then be compiled so as to form another signal which we will name S8 and which will be described later on;
e. Then, an algorithmic processing will be performed on each of the signals thus obtained so as to extract for each signal at least one estimated respiratory rate Rx.Ay per applied algorithm, x representing one of the processed signals and therefore being taken amongst at least 1, 2, 3, 4, 5, 6, 7, 8, and y corresponding to the algorithm used as described later on;
f. Then, based on these estimated respiratory rates Rx.Ay, a plurality of intermediate respiratory rates rx are computed, preferably r1, r2, r3 and r4;
g. Finally, the respiratory rate R of the user is computed by determining the median of the intermediate respiratory rates rx, such that R=median (r1, r2, r3, r4).

Thus, the present invention allows determining the respiratory rate of a user from a photoplethysmogram S(t) that could be obtained in various manners.

One of the numerous advantages of the present invention consists in the fact that the photoplethysmographic signal (PPG) represents the cardiac signal modulated by the respiratory signal. This PPG signal is very rich in information and the present invention allows exploiting this richness to extract one or several respiratory signal(s) on which respiratory rate estimates are then made.

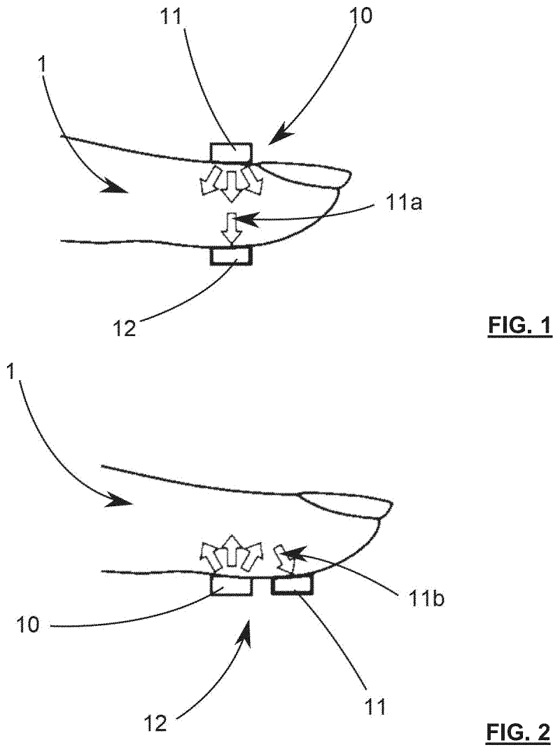

We will now describe a measuring device according to the present invention through FIGS. 1 and 2.

FIGS. 1 and 2 illustrate a measuring device 10 according to two embodiments of the present invention. This device 10 is disposed in contact with a finger 1 of a user.

For information, photoplethysmography is the technique used in oximetry, which is commonly used to measure arterial blood saturation (SpO2). Photoplethysmography provides non-invasive, continuous and real-time measurements of the signal representing the contour of the pulse wave with infrared light transmitted through a finger or a toe. This acquired signal is the digital pulse volume, a waveform that could provide elements on the health condition of a person, for example. The computation of a pulse wave has proved to be a reliable and reproducible technique to indirectly determine indices of arterial stiffness, for example. Hence, photoplethysmography is a useful non-invasive measurement of vascular dysfunction and of heart rate variability.

There are mainly, yet without restriction, two photoplethysmography modes: transmittance which is commonly used in oximetry and reflectance which is widely used in the measurement of heart rate by bracelets and smart watches for example. A light source transmits red and/or infrared light rays and an optical sensor captures the transmitted or reflected light.

It should be noted that for the present invention, a photoplethysmogram S(t) should be understood as a signal representing the variations of the digital volume of the pulse over time and/or of the respiratory rate of a user. Such a signal could be acquired via an optical sensor as well as via an electrical sensor, a radar sensor, a force sensor, a pressure sensor, a vibration sensor, an audio sensor, or still a seismic sensor. Preferably, it should be understood that according to the present invention, the word photoplethysmogram will also be used to refer to a plethysmogram.

In particular, it should be noted that via the present invention, the respiratory rate of a user could be assessed by the acquisition of data made by an optical sensor, an electrical sensor, a radar sensor, a force sensor, a pressure sensor, a vibration sensor, an audio sensor, or still a seismic sensor.

Indeed, the present invention applies to any type of photoplethysmograms acquired by various types of sensors such as those mentioned before, and/or acquired from a database of photoplethysmograms.

Advantageously, the data processing unit comprises at least one processor or a computer, and preferably a non-transitory memory. Preferably, the data processing unit may thus be a computer, a smart watch, a smartphone, a medical device, a computer server, etc. . . . . It should be noted that a data processing unit may also comprise a device for measuring a photoplethysmogram.

According to an embodiment illustrated in FIG. 1, the device 10 is disposed at least on either side of the finger 1 of the user. Advantageously, a light source 11 is disposed on one side of the finger 1 and an optical sensor 12 is disposed on the other side of the finger 1 so as to be able to capture the transmitted light rays 11a originating from the light source 11 and crossing the finger 1 of the user.

According to an embodiment illustrated in FIG. 2, the device 10 is disposed at least on one side of the finger 1 of the user. Advantageously, a light source 11 is disposed on one side of the finger 1 and an optical sensor 12 is disposed on the same side of the finger 1 as the light source 11 so as to be able to capture the reflected light rays 11b originating from the light source 11 and being reflected by the finger 1 of the user.

In each of these two embodiments, the measuring device 10 is configured to measure a photoplethysmogram S(t) 111 of the user.

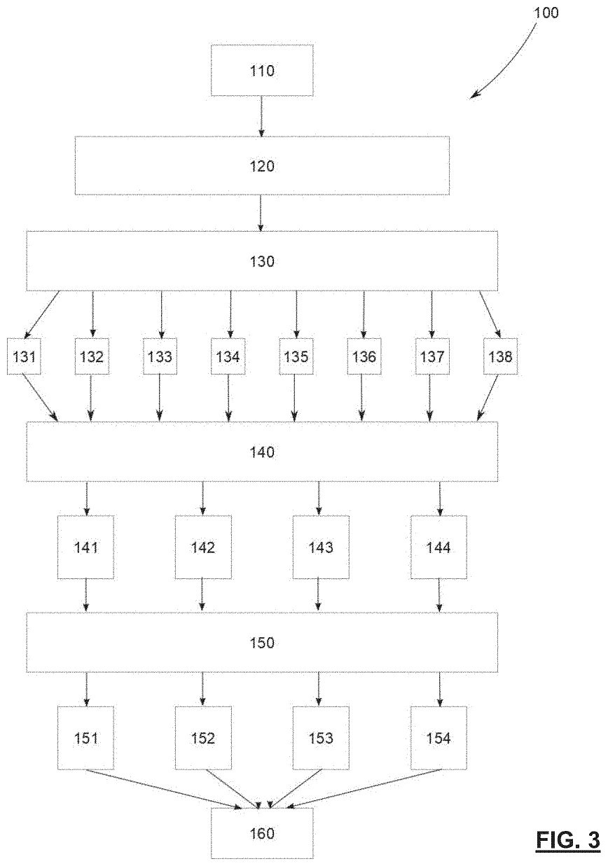

FIG. 3 illustrates a diagram schematising an embodiment of the method according to the present invention.

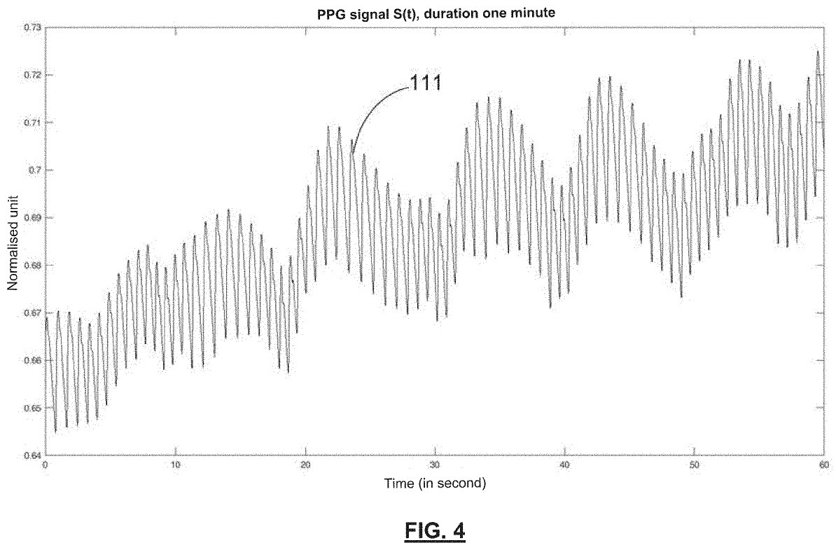

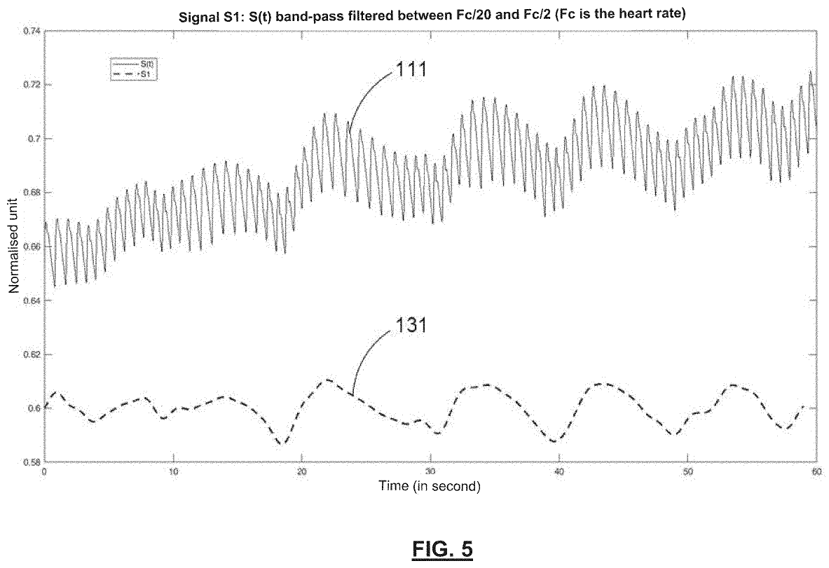

Thus, the method 100 for determining the respiratory rate R of at least one user comprising at least the following steps implemented by at least one data processing unit:
a. Acquisition 110 of at least one photoplethysmogram (called PPG) S(t) 111 of said user, preferably at a frequency higher than or equal to 50 Hz; The signal S(t) 111 is illustrated in FIG. 4 according to an embodiment of the present invention; In the present invention, by acquisition of at least one PPG it should be understood the fact of obtaining a PPG by measurement and/or the fact of receiving information from at least one measurement already carried out; Thus, according to one embodiment, the step 110 of acquiring said at least one photoplethysmogram (called PPG) S(t) 111 of said user is carried out by reception from at least one database of said photoplethysmogram S(t) 111 of said user;
b. Filtering 120 of S(t), preferably by a band-pass filter comprised between 0.5 Hz and 4 Hz, so as to generate a first filtered PPG signal Sf(t);
c. Estimation 120 of the heart rate Fc from Sf(t), preferably by identifying the maximum value of the power spectral density of Sf(t).
d. Filtering of Sf(t) between Fc/20 and 4 Hz so as to generate a second filtered photoplethysmogram S′f(t);
e. Obtainment 130 of at least three signals Sa, Sb, Sc distinct from each other, preferably at least 4, preferably at least 5, advantageously at least 6 and preferably at least 7, said distinct signals being taken amongst the signals S1, S2, S3, S4, S5, S6 and S7 described hereinafter;
f. Computation of an eighth signal 138 called S8 from said obtained signals;
g. Algorithmic processing 140 of each signal including S8 by at least two, preferably at least 3 and advantageously 4 algorithms A1, A2, A3 and A4 described later on, each algorithm being configured to generate at least one estimated respiratory rate per processed signal so as to obtain the estimated respiratory rates Rx.Ay corresponding to the estimated respiratory rate from the signal Sx and the algorithm Ay.
h. Computation 150 of at least two, preferably at least 3 and advantageously of 4 intermediate respiratory rates r1, r2, r3 and r4 as described later on.
i. Determination 160 of the respiratory rate R by finding the median of said intermediate respiratory rates.

As indicated before, and according to a preferred embodiment, all of the steps of the present method are carried out by a data processing unit of the computer type for example.

Advantageously, the present invention uses a plurality of processings of the PPG signal 111, as well as a plurality of algorithms to obtain an accurate and reliable value of the respiratory rate R of the user.

We will now detail the obtainment 130 of said signals S1 to S7 and the computation 138 of the signal S8.

Signal S1 131 is illustrated in FIG. 5. It is obtained by applying a band-pass filter between Fc/20 and Fc/2 to the second filtered photoplethysmogram S′f(t).

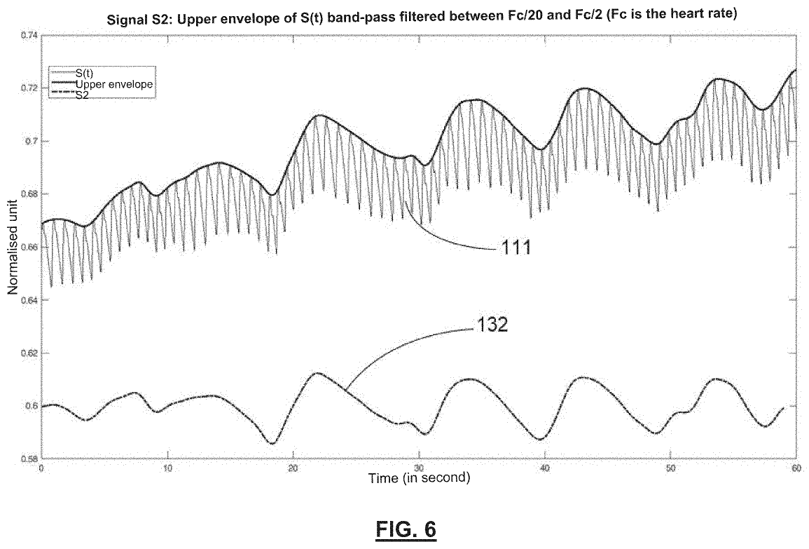

The signal S2 132 is illustrated in FIG. 6. It is obtained by:
a. determination of the peaks of the beats of the upper envelope of S′f(t),
b. then by filtering of the signal of the peaks of the beats of the upper envelope by a band-pass filter between Fc/20 and Fc/2.

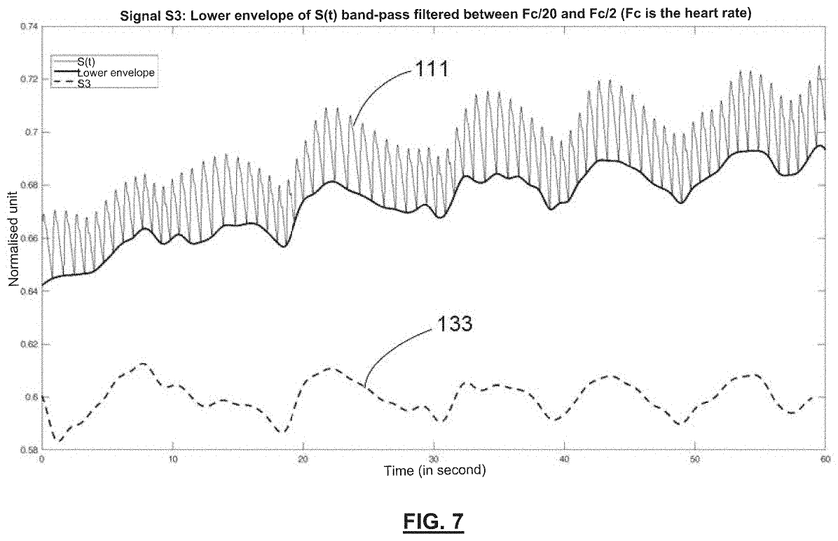

The signal S3 133 is illustrated in FIG. 7. It is obtained by:
a. determination of the valleys of the beats of the lower envelope of S′f(t),
b. then by filtering of the signal of the valleys of the beats of the lower envelope by a band-pass filter between Fc/20 and Fc/2.

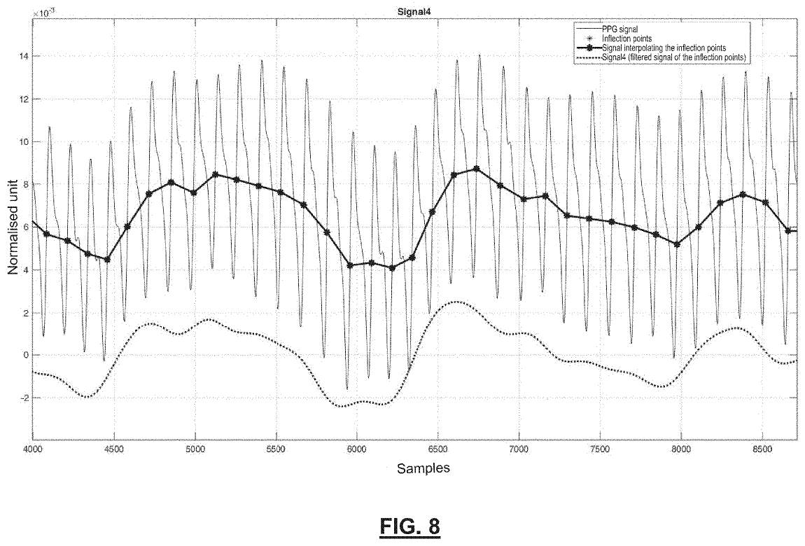

The signal S4 134 is illustrated in FIG. 8 as well as the original PPG signal S(t), S4 is then obtained by:
a. Generation of a pulse velocity wave VPG by performing a first-order derivative of the photoplethysmogram S(t) relative to the time coordinate of said photoplethysmogram S(t);
b. Determination of the positions Pi of the peaks of the pulse velocity wave VPG;
c. Obtainment of a variation signal of the inflection points by reporting values of said photoplethysmogram S(t) at the positions Pi;
d. Oversampling of the inflection point variation signal up to a predetermined frequency Fs, preferably generally equivalent to that of the signal S(t);
e. Band-pass filtering between the frequencies Fc/20 and Fc/2 of the oversampled signal.

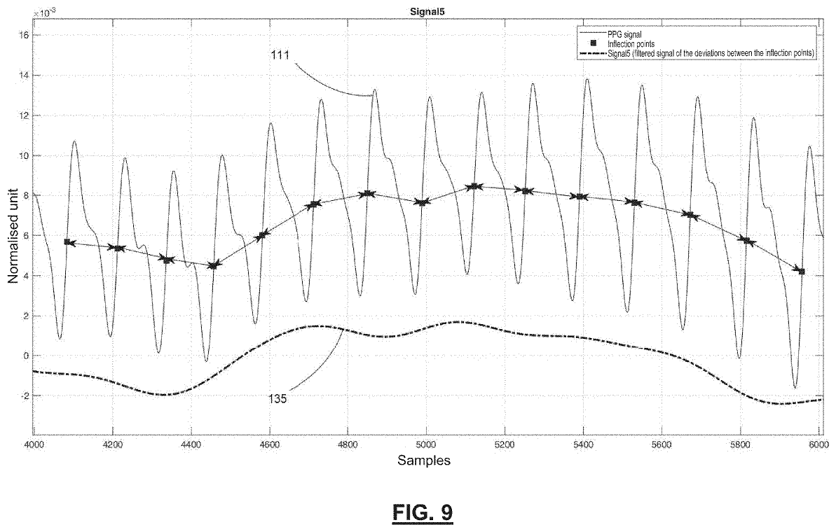

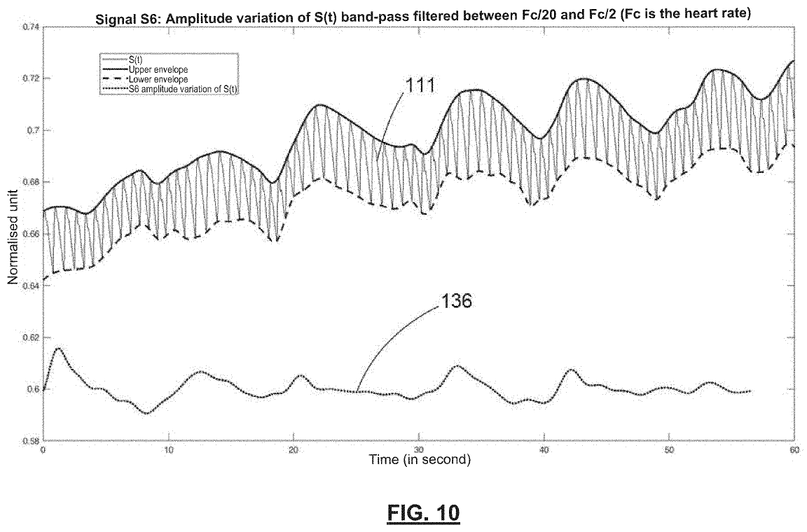

The signal S5 135 is illustrated in FIG. 9 as well as the original PPG signal S(t), S5 is then obtained by:
a. Location of the inflection points Pi in a systolic rise of a heartbeat of a user by identifying the position of the peaks of the first-order derivative of S(t) relative to the time coordinate of S(t)
b. Obtainment of a variation signal of the inflection points Pi by reporting values of time differences between Pi and Pi−1;
c. Oversampling of this signal of intervals of the inflection points Pi up to a predetermined frequency Fs, preferably generally of the same order as S(t);
d. Band-pass filtering between frequencies Fc/20 and Fc/2 of the oversampled signal;

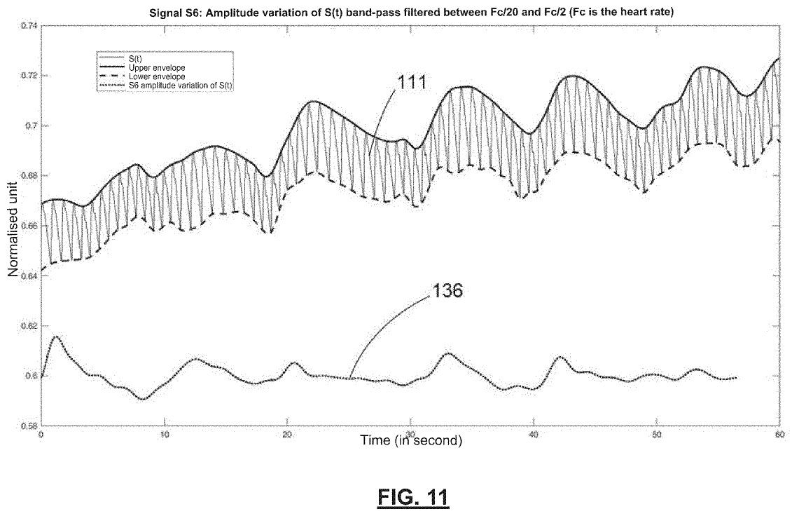

The signal S6 136 is illustrated in FIG. 10. It is obtained by:
a. determination of the amplitude variations of S′f(t),
b. then by filtering by a band-pass filter between Fc/20 and Fc/2 of the amplitude variation signal.

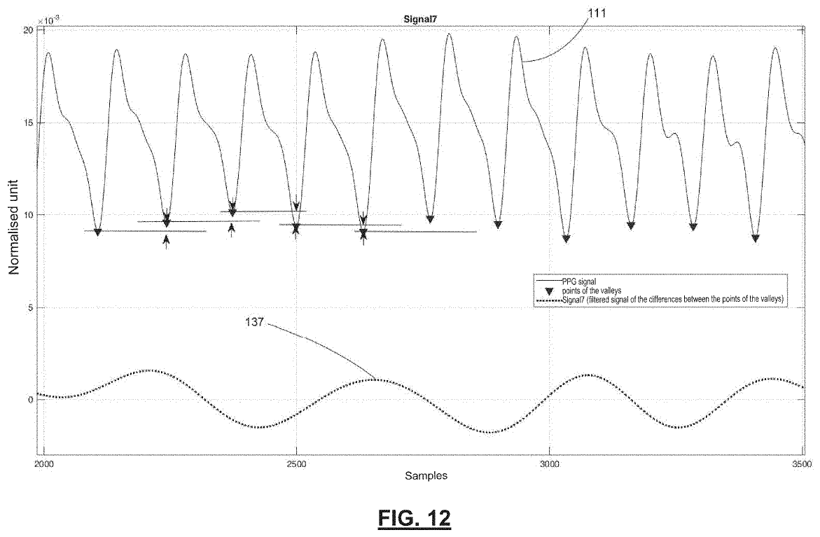

The signal S7 137 is illustrated in FIG. 11 as well as the original PPG signal S(t), S4 is then obtained by:
a. determination of the valleys of the beats of the lower envelope of the second filtered photoplethysmogram,
b. oversampling of the signal of the valleys of the beats of the lower envelope up to a predetermined frequency Fs, preferably generally of the same order as the frequency of the signal S(t);
c. First-order differentiation according to the time coordinate of the oversampled signal;
d. then by filtering of the derived signal by a band-pass filter between Fc/20 and Fc/2.

The signal S8 138 is illustrated in FIG. 12 as well as the autocorrelation signals of S1, S2, S3, S4, S5, S6 and S7. The signal S8 is generated from at least 3, preferably 4, advantageously 5, preferably 6 and advantageously 7 signals S1, S2, S3, S4, S5, S6 and S7. S8 is preferably generated by the following steps:
a. Computation respectively for each signal Sx of its normalised autocorrelation signal Sxc;
b. The signal S8 is equal to the arithmetic average of the normalised autocorrelation signals Sxc.

In a particularly advantageous manner, some of said signals S1 to S8 have a tendency to better represent slow and deep breathing (like S1, S2, S3 for example) and others better represent rapid and shallow breathing (like S5, S6 and S7 for example). Combining the signals harmonises these effects. In addition, this combination of signals to obtain S8 uses the autocorrelation function thereby allowing highlighting the natural undulations of breathing present in the cardiac signal.

According to a preferred embodiment, all of the computations and signal processing of the present invention are carried out by the data processing unit.

We will now describe the previously discussed algorithms A1, A2, A3 and A4.

Advantageously, these algorithms are configured to be applied to each signal S1 to S8. For simplicity reasons, in the next paragraphs, Sx refers to any signal taken amongst at least S1 to S8.

According to one embodiment, the algorithm A1 141 comprises at least the following steps applied to the signal Sx:
a. Collection of the measurement points corresponding to a crossing of the abscissas by the considered signal, i.e. Sx(t)=0;
b. Computation of the average interval Tm between each of the abscissa crossing points;
c. Computation of the estimated respiratory rate Rx.A1 with Rx.A1=60/(2*Tm);

As a non-limiting example, the algorithm A1 141 applied to the signal S1 produces an estimated respiratory rate R1.A1, for the signal S2 this will be R2.A1, and so on for the other signals.

According to one embodiment, the algorithm A2 142 comprises at least the following steps applied to the signal Sx:
a. Collection of the measurement points corresponding to the peak of the undulations of the considered signal;
b. Computation of the average interval Ts between each of the peak points;
c. Computation of the estimated respiratory rate Rx.A2 with Rx.A2=60/(Ts).

As a non-limiting example, the algorithm A2 142 applied to the signal S4 produces an estimated respiratory rate R4.A2, for the signal S3 this will be R3.A2, and so on for the other signals.

According to one embodiment, the algorithm A3 143 comprises at least the following steps applied to the signal Sx:
a. Determination of the correntropy spectral density by computing the Fourier transform of the centred autocorrentropy of the considered signal;
b. Extraction of the estimated respiratory rate Rx.A3 by determining the peak of the correntropy spectral density over a predetermined frequency range, preferably between max(Fc/20, 0.05) and min(Fc/2.1) where 0.05 Hz and 1 Hz respectively correspond to respiratory rates of 3 and 60 breaths per minute

As a non-limiting example, the algorithm A3 143 applied to the signal S7 produces an estimated respiratory rate R7.A3, for the signal S8 this will be R8.A3, and so on for the other signals.

According to one embodiment, the algorithm A4 144 comprises at least the following steps applied to the signal Sx:
a. Computation of the autocorrelation signal of the considered signal;
b. Collection of the measurement points corresponding to the peak of the undulations of the autocorrelation signal of the considered signal, preferably corresponding to the peak of the undulations of the second half of the autocorrelation signal of the considered signal;
c. Computation of the average interval Tas between each of the peak points;
d. Computation of the estimated respiratory rate Rx.A4 with Rx.A4=60/(Tas).

As a non-limiting example, the algorithm A4 144 applied to the signal S5 produces an estimated respiratory rate R5.A4, for the signal S6 this will be R6.A4, and so on for the other signals.

In a particularly advantageous manner, these algorithms are very complementary in terms of performance: if the respiratory signal is of very good quality, the four algorithms will converge towards the same result. Conversely, if the respiratory signal features artefacts, the algorithms A1, A2, A3 and A4 will have different sensitivities that could be used to minimise the error in estimating the final respiratory rate.

According to a preferred embodiment, all of the algorithms A1, A2, A3 and A4 are implemented by the data processing unit. According to one embodiment, at least part of these algorithms could be implemented via a computer server.

We will now describe the computation of the respiratory rates r1, r2, r3 and r4 from at least part of the previously estimated respiratory rates Rx.Ay.

The advantage of using the computation of the median on subsets of estimated respiratory rates is to minimise the error rate if half of these rates diverge from the true value. Unlike the average which will proportionally keep the effect of this divergence in the final result.

According to one embodiment, the first intermediate respiratory rate r1 151 is equal to the median of the medians of the estimated respiratory rates Rx.Ay of a signal Sx, Sx being taken amongst at least S1 to S7.

Thus, in the case where all of the signals S1 to S7 are considered, r1=median (median ((R1.A1, R1.A2, R1.A3, R1.A4)), median ((R2.A1, R2.A2, R2.A3, R2.A4)), median ((R3.A1, R3.A2, R3.A3, R3.A4)), median ((R4.A1, R4.A2, R4.A3, R4.A4)), median ((R5.A1, R5.A2, R5.A3, R5.A4)), median ((R6.A1, R6.A2, R6.A3, R6.A4)), median ((R7.A1, R7.A2, R7.A3, R7.A4))).

According to one embodiment, the second intermediate respiratory rate r2 152 corresponding to the top of the histogram formed by the estimated respiratory rates Rx.Ay of each considered signal Sx taken amongst S1 to S7 processed throughout each considered algorithm Ay taken amongst A1 to A4.

Thus, for example, considering the set of signals S1 to S7 and the set of algorithms A1 to A4, r2 corresponds to the top of the histogram formed by the following values: R1.A1, R1.A2, R1.A3, R1.A4, R2.A1, R2.A2, R2.A3, R2.A4, R3.A1, R3.A2, R3.A3, R3.A4, R4.A1, R4.A2, R4.A3, R4.A4, R5.A1, R5.A2, R5.A3, R5.A4, R6.A1, R6.A2, R6.A3, R6.A4, R7.A1, R7.A2, R7.A3, R7.A4.

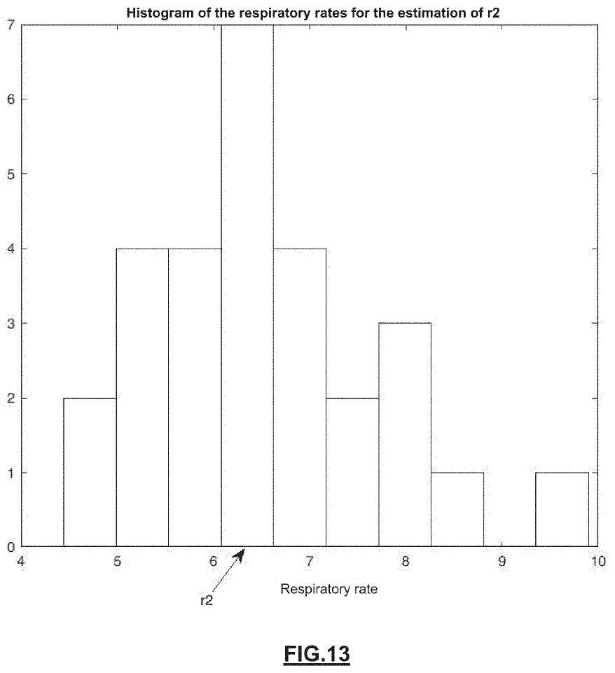

FIG. 13 illustrates such a histogram according to a non-limiting example.

According to one embodiment, the third intermediate respiratory rate r3 153 is equal to the median value of the estimated respiratory rates for each considered signal processed by the third algorithm A3.

Thus, for example, in the case where all of the signals S1 to S7 are considered, r3=median (R1.A3, R2.A3, R3.A3, R4.A3, R5.A3, R6.A3, R7.A3).

According to one embodiment, the fourth intermediate respiratory rate r4 154 is equal to the median value of the estimated respiratory rates of the signal S8.

Thus, for example, in the case where the set of algorithms A1 to A4 processes the signal S8, r4=median (R8.A1, R8.A2, R8.A3, R8.A4).

Afterwards, as indicated before, the respiratory rate R of the user is computed by finding the median of at least part of the intermediate respiratory rates computed before. Herein again, all of the computations implemented by the method of the present invention are preferably executed by the data processing unit.

Thus, the present invention allows accurately and reliably determining the respiratory rate of a user by simply measuring his heart rate and in particular a photoplethysmogram signal S(t).

The processing and algorithms then applied to this signal allow reducing the noise of the measurements and exploiting a simple and inexpensive measurement to accurately and reliably determine the respiratory rate of a user, and that being so in a very short time.

The invention is not limited to the previously-described embodiments and extends to all embodiments covered by the claims.

### REFERENCES

1 Finger of the user
10 Measuring device
11 Light source
11a Transmitted light rays
11b Reflected light rays
12 Optical sensor
100 Method for determining the respiratory rate R
110 Capture of the photoplethysmogram signal S(t)
111 Photoplethysmogram signal S(t)
120 Filtering of the photoplethysmogram S(t) and estimation of the heart rate Fc
130 Obtainment of 8 signals
131 Signal S1
132 Signal S2
133 Signal S3
134 Signal S4
135 Signal S5
136 Signal S6
137 Signal S7
138 Signal S8
140 Algorithmic processing of the signal
141 First algorithm
142 Second algorithm
143 Third algorithm
144 Fourth algorithm
150 Computation of the intermediate respiratory rates
151 First intermediate respiratory rate r1
152 Second intermediate respiratory rate r2
153 Third intermediate respiratory rate r3
154 Fourth intermediate respiratory rate r4
160 Computation of the respiratory rate R

1. A method for determining a respiratory rate R of at least one user comprising at least the following steps implemented by at least one data processing unit comprising:
a. Acquisition of at least one photoplethysmogram S(t) of said user, the step of acquiring said photoplethysmogram S(t) comprising at least one amongst the following steps:
Measurement, preferably at a frequency higher than or equal to 50 Hz, of said at least one photoplethysmogram S(t) by a measuring device, preferably worn or intended to be worn by said at least one user, the measuring device comprising at least one sensor adapted to measure at least one photoplethysmogram S(t);
Reception from at least one database of said at least one photoplethysmogram S(t);
b. Estimation of a heart rate Fc from said at least one photoplethysmogram S(t) acquired from said at least one user;
c. Obtainment of at least three signals distinct from each other, each obtained signal being a function of said at least one acquired photoplethysmogram S(t);
d. Generation of a signal S8 from said at least three obtained signals;
e. Algorithmic processing of each of the at least three obtained signals and of the signal S8 by at least two algorithms Ai and Aj, so as to obtain at least two estimated respiratory rates, respectively Rx.Ai and Rx.Aj, for each signal Sx processed respectively by the algorithm Ai and by the algorithm Aj, each signal Sx being taken amongst said at least three obtained signals and the signal S8; the at least two algorithms Ai and Aj being distinct from each other;
f. Computation of at least two intermediate respiratory rates rm and rn from the estimated respiratory rates;
g. Determination of the respiratory rate R by computing a median of said at least two intermediate respiratory rates rm and rn.

2. The method according to claim 1, comprising, before the step of estimating the heart rate Fc, a step of filtering said photoplethysmogram, preferably by a band-pass filter, so as to generate a first filtered photoplethysmogram.

3. The method according to claim 2, comprising, after the step of estimating the heart rate Fc, an additional step of filtering said first photoplethysmogram filtered between Fc/20 and 4 Hz so as to generate a second filtered photoplethysmogram

4. The method according to claim 3, wherein said at least three obtained distinct signals are taken amongst at least the following signals S1, S2, S3, S4, S5, S6 and S7:
a. a signal S1 obtained by
Application of a band-pass filter between Fc/20 and Fc/2 to the second filtered photoplethysmogram.
b. a signal S2 obtained by:
determination of peaks of beats of an upper envelope of the second filtered photoplethysmogram,
then by filtering of the signal of the peaks of the beats of the upper envelope by a band-pass filter between Fc/20 and Fc/2.
c. a signal S3 obtained by:
determination of valleys of beats of a lower envelope of the second filtered photoplethysmogram;
then by filtering of the signal of the valleys of the beats of the lower envelope by a band-pass filter between Fc/20 and Fc/2;
d. a signal S4 obtained by:
Generation of a pulse velocity wave VPG by performing a first-order derivative of the photoplethysmogram S(t) relative to the time coordinate of said photoplethysmogram S(t);
Determination of the positions Pi of peaks of the pulse velocity wave VPG;
Obtainment of a signal of variation of inflection points by reporting values of said photoplethysmogram S(t) at the positions Pi;
Oversampling of the signal of variation of the inflection points up to a predetermined frequency Fs;
Band-pass filtering between the frequencies Fc/20 and Fc/2 of the oversampled signal;
e. a signal S5 obtained by:
Location of inflection points Pi in a systolic rise of a heartbeat of the user by identifying the position of peaks of the first-order derivative of the photoplethysmogram S(t) relative to the time coordinate of said photoplethysmogram S(t)
Obtainment of a signal of variation of the inflection points Pi by reporting values of time differences between Pi and Pi−1;
Oversampling of this signal of intervals of the inflection points Pi up to a predetermined frequency Fs;
Band-pass filtering between the frequencies Fc/20 and Fc/2 of the oversampled signal;
f. a signal S6 obtained by:
determination of amplitude variations of the second filtered photoplethysmogram,
then by filtering by a band-pass filter between Fc/20 and Fc/2 of the amplitude variation signal;
g. a signal S7 obtained by:
determination of the valleys of the beats of the lower envelope of the second filtered photoplethysmogram,
oversampling of the signal of the valleys of the beats of the lower envelope up to a predetermined frequency Fs;
First-order differentiation according to the time coordinate of the oversampled signal;
then by filtering of the derived signal by a band-pass filter between Fc/20 and Fc/2.

5. The method according to claim 1, wherein the step of generating a signal S8 from said at least three obtained signals comprises at least the following steps:
a. Computation, for each signal amongst said at least three obtained signals, of a normalised autocorrelation signal;
b. Computation of an arithmetic average of the normalised autocorrelation signals.

6. The method according to claim 1, wherein the at least two algorithms Ai and Aj distinct from each other are taken amongst at least the following algorithms A1, A2, A3 and A4:
a. an algorithm A1 comprising at least the following steps applied to the signal Sx:
Collection of measurement points corresponding to a crossing of the abscissas by the considered signal, i.e. Sx(t)=0;
Computation of the average interval Tm between each of the abscissa crossing points;
Computation of Rx.A1 with Rx.A1=60/(2*Tm);
b. an algorithm A2 comprising at least the following steps applied to the signal Sx:
Collection of measurement points corresponding to a peak of the undulations of the considered signal;
Computation of an average interval Ts between each of the peak points;
Computation of Rx.A2 with Rx.A2=60/(Ts);
c. an algorithm A3 comprising at least the following steps applied to the signal Sx:
Determination of a correntropy spectral density by computing the Fourier transform of a centred autocorrentropy of the considered signal;
Extraction of Rx.A3 by determining a peak of the correntropy spectral density over a predetermined frequency range;
d. an algorithm A4 comprising at least the following steps applied to the signal Sx:
Computation of an autocorrelation signal of the considered signal;
Collection of measurement points corresponding to a peak of undulations of the autocorrelation signal of the considered signal;
Computation of an average interval Tas between each of the peak points;
Computation of Rx.A4 with Rx.A4=60/(Tas);

7. The method according to claim 6, wherein the at least two intermediate respiratory rates rm and rn are taken amongst at least the following intermediate respiratory rates r1, r2, r3 and r4:
a. r1 is equal to a median value of the estimated respiratory rates Rx.Ay of a signal Sx, Sx being taken amongst at least the obtained signals;
b. r2 corresponds to the top of the histogram formed by the estimated respiratory rates Rx.Ay of each considered signal Sx taken amongst the obtained signals processed throughout each considered algorithm Ay taken amongst A1 to A4;
c. r3 is equal to a median value of the estimated respiratory rates Rx.A3 for each considered signal Sx taken amongst the obtained signals processed by the algorithm A3;
d. r4 is equal to a median value of the estimated respiratory rates R8.Ay of the signal S8 processed throughout each considered algorithm Ay taken amongst A1 to A4.

8. The method according to claim 1, comprising a step of acquiring said at least one photoplethysmogram S(t) of said user.

9. The method according to claim 1, wherein the step of obtaining at least three signals distinct from each other comprises the obtainment of at least 4, preferably at least 5, advantageously at least 6 and advantageously of the 7 distinct signals S1, S2, S3, S4, S5, S6 and S7.

10. The method according to claim 6, wherein the algorithmic processing step comprises the use of at least 3 and preferably of the 4 distinct algorithms A1, A2, A3 and A4 applied to each of the obtained signals and of the signal S8.

11. The method according to claim 9, wherein r1=median (median ((R1.A1, R1.A2, R1.A3, R1.A4)), median ((R2.A1, R2.A2, R2.A3, R2.A4)), median ((R3.A1, R3.A2, R3.A3, R3.A4)), median ((R4.A1, R4.A2, R4.A3, R4.A4)), median ((R5.A1, R5.A2, R5.A3, R5.A4)), median ((R6.A1, R6.A2, R6.A3, R6.A4)), median ((R7.A1, R7.A2, R7.A3, R7.A4))).

12. The method according to claim 7, wherein r2 corresponds to the top of the histogram formed by the following values: R1.A1, R1.A2, R1.A3, R1.A4, R2.A1, R2.A2, R2.A3, R2.A4, R3.A1, R3.A2, R3.A3, R3.A4, R4.A1, R4.A2, R4.A3, R4.A4, R5.A1, R5.A2, R5.A3, R5.A4, R6.A1, R6.A2, R6.A3, R6.A4, R7.A1, R7.A2, R7.A3, R7.A4.

13. The method according to claim 7, wherein r3=median (R1.A3, R2.A3, R3.A3, R4.A3, R5.A3, R6.A2, R7.A3).

14. The method according to claim 7, wherein r4=median(R8.A1, R8.A2, R8.A3, R8.A4).

15. The method according to claim 11, wherein R=median (r1, r2, r3, r4).

16. The method according to claim 1, wherein S8=(S1c+S2c+S3c+S4c+S5c+S6c+S7c)/7, with the signals Sxc corresponding to the normalised autocorrelation signal of the signal Sx taken amongst S1, S2, S3, S4, S5, S6 and S7.

17. A computer program product, preferably recorded on a non-transitory medium, comprising instructions, which when performed by at least one amongst a processor and a computer, result in that the at least one amongst the processor and the computer, executes the method according to claim 1.

18. A device for measuring a photoplethysmogram S(t) of a user connected to at least one data processing unit comprising at least one non-transitory memory comprising a computer program product according to claim 17, the measuring device comprising at least one sensor adapted to measure a photoplethysmogram.

19. The device according to claim 18, comprising a module for wireless or wired communication with said data processing unit.

20. The device according to claim 18, wherein the sensor adapted to measure a photoplethysmogram is a sensor taken amongst at least: an optical sensor, an electrical sensor, a radar sensor, a force sensor, a pressure sensor, a vibration sensor, an audio sensor, a seismic sensor.

21. The device according to claim 18, comprising at least one sensor of the respiratory rate of said user taken amongst at least: an optical sensor, an electrical sensor, a radar sensor, a force sensor, a pressure sensor, a vibration sensor, an audio sensor, a seismic sensor.
